(19) United States
(12) Patent Application Publication
(10) Pub. No.: US 2024/0382142 A1
(43) Pub. Date: Nov. 21, 2024

(54) A SYSTEM, COMPUTER PROGRAM AND METHOD

(71) Applicant: Sony Group Corporation, Tokyo (JP)

(72) Inventors: Naoyuki Hirota, Tokyo (JP); David Duffy, London (GB); Christopher Wright, London (GB)

(73) Assignee: Sony Group Corporation, Tokyo (JP)

(21) Appl. No.: 18/686,544

(22) PCT Filed: Jun. 16, 2022
(86) PCT No.: PCT/JP2022/024195

§ 371 Date: Feb. 26, 2024

(30) Foreign Application Priority Data
Sep. 10, 2021 (EP) 21196093.5

(51) Int. Cl. A61B 5/00; A61B 5/11; A61B 5/389; G16H 50/20; G16H 50/30

(52) U.S. Cl. CPC A61B 5/4088; A61B 5/11; A61B 5/389; A61B 5/6802; G16H 50/20; G16H 50/30

(57) ABSTRACT

A system for determining an indicator value of cognitive impairment in a user comprising circuitry configured to: receive motion data from a wearable device worn by the user during a time period comprised of a first time period and a second time period, the second time period being after the first time period; compare the motion data received during the second time period with stored reference motion data, the stored reference motion data representing reference motion carried out when performing a predetermined task during the second period of time; determine the predetermined task based upon the comparison; compare the motion data received during the first time period with further stored reference motion data, the further stored reference motion data representing reference motion carried out when performing the determined predetermined task during the first time period; and determine the indicator value of the cognitive impairment based upon a comparison between the motion data received during the first time period with the further reference motion data.

### TECHNICAL FIELD

The present technique relates to a system, computer program and method.

### BACKGROUND

The “background” description provided herein is for the purpose of generally presenting the context of the disclosure. Work of the presently named inventors, to the extent it is described in the background section, as well as aspects of the description which may not otherwise qualify as prior art at the time of filing, are neither expressly or impliedly admitted as prior art against the present technique.

Motor planning is the cognitive skill of executing motions with body parts to perform an action, such as picking up an object, turning a door handle or pushing a button. Motor planning errors are indicated by a declining ability to perform these motions smoothly.

Motor planning errors have been broadly correlated with early cognitive decline, indicative of both late-age-related cognitive decline (e.g. people 70+ years old) and disorders such as Alzheimer's and mild cognitive impairment (MCI). Most studies have focused on the presentation of the motor planning errors in specific fine motor control tasks (such as finger tasks), as these are the tasks for which large disruptions are most evident.

However, recently a group demonstrated that MCI could be determined from coarse motions (large limb motion) using just the accelerometer data from a wearable device. In a task where the user had to move their leg, the acceleration curve of from the accelerometer could be used to identify precisely any motor planning error, and its severity.

Specifically, it was shown that a healthy curve would have a peak acceleration in the middle of the motion, as a smooth curve, but the MCI curve would be less smooth, with more variation in acceleration.

At present, these tests for MCI are carried out in a clinical setting requiring the individual under test to attend a particular location and to perform these tests under clinical supervision. This is very inconvenient for the individual. Moreover, as the tests are carried out in a clinical setting, only a small number of tests can be carried out every day. This means that testing for an individual will be infrequent and so continual monitoring of an individual's symptoms is not possible.

Other prior art is provided in WO2014143896 and US20170229037.

It is an aim of the disclosure to address at least one of these issues.

### SUMMARY

Embodiments of the disclosure are defined by the appended claims.

The foregoing paragraphs have been provided by way of general introduction, and are not intended to limit the scope of the following claims. The described embodiments, together with further advantages, will be best understood by reference to the following detailed description taken in conjunction with the accompanying drawings.

### BRIEF DESCRIPTION OF DRAWINGS

A more complete appreciation of the disclosure and many of the attendant advantages thereof will be readily obtained as the same becomes better understood by reference to the following detailed description when considered in connection with the accompanying drawings.

FIG. 1 shows a Mild Cogitative Impairment (MCI) Risk Assessment device 100, an MCI Risk Notification device 200 and an Inertial Measurement device 300 according to embodiments of the present disclosure.

FIG. 2 shows a functional diagram of the MCI Risk Assessment device 100, the MCI Risk Notification device 200 and the Inertial Measurement device 300 according to embodiments of the present disclosure.

FIG. 3 shows the functional diagram of FIG. 2 in more detail.

FIG. 4 shows a process according to embodiments of the disclosure.

FIG. 5 shows a known Motor Task Diagram.

FIG. 6 explains the Motor Task Indicator Algorithm shown in FIGS. 2 and 3.

FIG. 7 explains the IMU Data Motion Assessment Algorithm shown in FIGS. 2 and 3.

FIG. 8 shows real-world use cases according to embodiments of the disclosure.

FIG. 9 shows a process explaining Musculoskeletal Disorder Differentiation.

FIG. 10 shows a process explaining Advanced Cognitive Disorder Differentiation.

FIG. 11 shows a system diagram according to embodiments of the disclosure.

### DESCRIPTION OF EMBODIMENTS

Referring now to the drawings, wherein like reference numerals designate identical or corresponding parts throughout the several views.

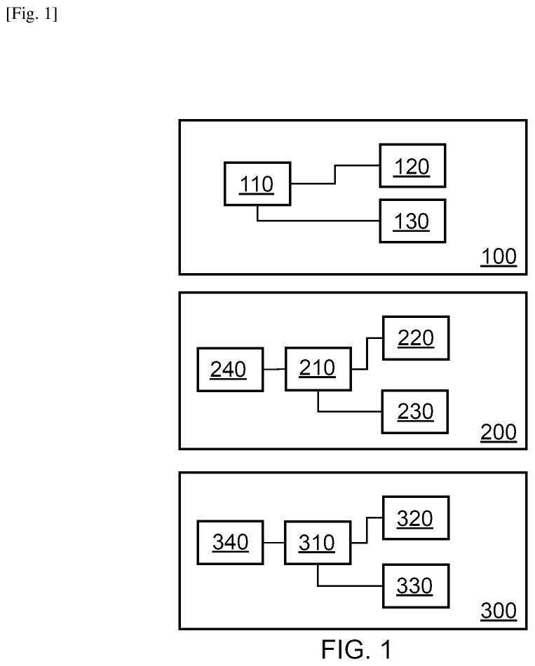

In FIG. 1 an MCI Risk Assessment device 100, an MCI Risk Notification device 200 and an Inertial Measurement device 300 according to embodiments of the present disclosure is shown. For the sake of brevity, the MCI Risk Assessment device 100 will be referred to as the MCI RA device 100, the MCI Risk Notification device 200 will be referred to as the MCI RN device 200 and the Inertial Measurement device 300 will be referred to as the IMU 300.

As will be explained later, the MCI RA device 100 determines the risk that an individual has MCI and if the individual has MCI, the severity of the MCI. In other words, the MCI RA device 100 screens or detects long-term MCI risk in an individual by processing IMU Data to detect and analyse particular motions (i.e. the Motor Task Interaction Phase motions) for MCI symptoms or severity. This will be explained later. This is achieved by the MCI RA device 100 (as part of a system) determining an indicator value of the cognitive impairment. This indicator value is indicative of the risk and/or severity of the cognitive impairment and in embodiments indicates a change in cognitive impairment over a period of time.

Depending upon the risk or severity of MCI will determine whether the individual and/or his or her medical practitioner caring for that individual will be notified of the risk or severity of the individual's MCI. The notification will be carried out by the MCI RN device 200. Finally, the MCI RA device 100 will receive motion data which is the motion carried out by the individual when performing various tasks from the IMU 300.

The MCI RA device 100 includes MCI RA processing circuitry 110. Connected to the MCI RA processing circuitry 110 is MCI RA storage 120 and MCI RA communication circuitry 130. In embodiments, the MCI RA processing circuitry 110 is any kind of circuitry such as programmable semiconductor circuitry (for example an Application Specific Integrated Circuit “ASIC”) or a processor that is controlled using computer software. The computer software is stored on the MCI RA storage 120. Moreover, as will become apparent, other data such as various databases are stored on the MCI RA storage 120. The MCI RA communication circuitry 130 is provided to communicate either via a wired or wireless connection with the MCI RN device 200 and/or the IMU 300. The connection may be using one of many Standards such as the Bluetooth Standard or an Ethernet Standard.

The MCI RN device 200 includes MCI RN processing circuitry 210. Connected to the MCI RN processing circuitry 210 is MCI RN storage 220 and MCI RN communication circuitry 230. In addition, the MCI RN device 200 includes an MCI RN display 240 connected to the MCI RN processing circuitry 210. This provides feedback to the individual and/or his or her medical practitioner caring for the individual. Although in embodiments this feedback is visual, the disclosure is not so limited and any kind of audio and/or haptic feedback may be provided in addition to or instead of the visual feedback.

In embodiments, the MCI RA processing circuitry 110 is any kind of circuitry such as programmable semiconductor circuitry (for example an Application Specific Integrated Circuit “ASIC”) or a processor that is controlled using computer software. The computer software is stored on the MCI RN storage 220. Moreover, as will become apparent, other data such as various databases are stored on the MCI RN storage 220. The MCI RN communication circuitry 230 is provided to communicate either via a wired or wireless connection with the MCI RA device 100 and/or the IMU 300. The connection may be using one of many Standards such as the Bluetooth Standard or an Ethernet Standard.

The IMU device 300 includes IMU processing circuitry 310. Connected to the IMU processing circuitry 310 is IMU storage 320 and IMU communication circuitry 330. In addition, the IMU device 300 includes sensor circuitry 340 connected to the IMU processing circuitry 310. This provides motion data indicative of the movement of one or more body parts of the individual. In embodiments, this motion data is collected continuously or periodically or upon instruction from the individual and may be acceleration and/or velocity data that may be multidimensional. In other words, the acceleration and/or velocity data may be collected in one, two or three dimensions over time. In embodiments, the IMU device may be worn on a limb such as a watch, patch, ring or the like or may be a handheld device such as a smartphone (held during use), earphone (held to be placed in cars), AR glasses (held to be placed on the head), or the like. In embodiments, the IMU device may be worn or handled in one or more body locations. Specifically, for the purposes of assessing Motor Planning Error (which as will be explained is used to calculate MCI), body locations may include those which can monitor motion of the hand and wrist such as: attached to wrist (e.g. worn mSafety watch); attached to fingers or hand (e.g. smart ring); and/or held in the hand (e.g. earphones, AR glasses, or the like). Of course the disclosure is not so limited and the IMU device 300 may be worn in any other additional body location, such as the foot/ankle, torso or head.

In embodiments, the IMU processing circuitry 310 is any kind of circuitry such as programmable semiconductor circuitry (for example an Application Specific Integrated Circuit “ASIC”) or a processor that is controlled using computer software. The computer software is stored on the IMU storage 320. Moreover, as will become apparent, other data such as various databases are stored on the IMU storage 320. The IMU communication circuitry 330 is provided to communicate either via a wired or wireless connection with the MCI RA device 100 and/or the MCI RN device 200. The connection may be using one of many Standards such as the Bluetooth Standard or an Ethernet Standard.

It should be noted that the following describes the IMU device 300 capturing the motion data. However, the disclosure is not so limited and some IMU devices 300, such as handheld versions of IMU devices 300, may only be useful for providing data to assess Motor Planning Error in combination with other devices. For example, handheld devices may not characterise completely a Motor Task motion, as they are picked up or released only partway through the motion-in this case, other devices (such as wearable devices) may be used in tandem to collect all necessary data.

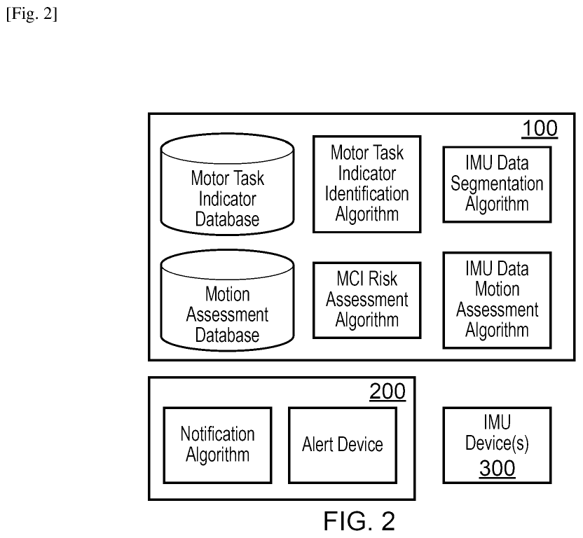

FIG. 2 shows a functional diagram of the MCI RA device 100, the MCI RN device 200 and the IMU device 300 according to embodiments of the present disclosure.

In the MCI RA device 100, various algorithms are carried out by the MCI RA processing circuitry 110. These are the Motor Task Indicator Identification algorithm, the MCI Risk Assessment algorithm, the IMU Data Segmentation Algorithm and the IMU Data Motion Assessment Algorithm. The computer program code used to carry out these algorithms is stored in the MCI RA storage 120. In addition, the MCI RA storage 120 includes a motor task indicator database and a motion assessment database. These algorithms and databases will now be described.

The Motor Task Indicator Database contains acceleration waveforms that are indicative of a Motor Task.

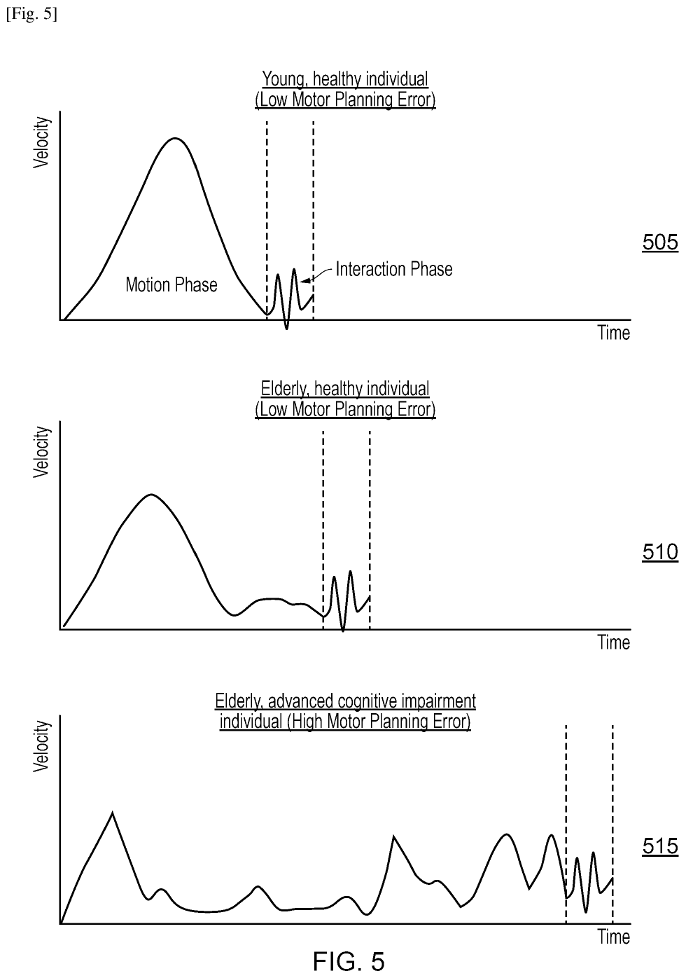

A Motor Task is a task that that requires a user to move a body part from one position to another to interact in some way with their environment, such as picking up a cup, grabbing a door handle, or placing earphones in their cars or the like. The Motor Task is defined by the following three phases:
1. Motion Phase-the part of the Motor Task where the user moves their body part from the first position to the position of the object they want to interact with (e.g. moving hand to position of a cup). The Motion Phase is motion carried out during a first time period.
   i. In a healthy individual (or an individual with low MCI), body part acceleration during the Motion Phase will follow a controlled smooth curve of constantly changing acceleration, starting from 0, reaching maximum velocity in the middle of the motion, and then returning to 0 once the user reaches the goal. This is shown as the “Motion Phase” in graph 505 of FIG. 5.
   ii. In an individual expressing MCI symptoms, body part acceleration during the Motion Phase will be less smooth with the maximal velocity being less central, and may have step changes in acceleration, direction reversals, etc. This is shown in the left hand part of graph 515 in FIG. 5.
2. Interaction Phase-the part of the Motor Task where the user performs some interaction with the object (e.g. grabbing the cup). This is carried out during a second time period that is after the first time period (i.e. after the motion phase). This Interaction Phase may involve a characteristic hand and/or finger position (e.g. a grip posture, a finger pointing posture, a pinching posture), with a corresponding IMU data signature (referred to as the reference motion hereinafter).
3. Motor Planning Error-the deviation from ideal motion during the Motion Phase.
   i. A low or very low Motor Planning Error (i.e. close to the motion shown in the “Motion Phase” in graph 505 of FIG. 5) will result in a smooth acceleration curve in the Motion Phase waveform, a higher peak velocity, and a faster overall Motion Phase.
   ii. High Motor Planning Error (i.e. deviation from the ideal motion such as that shown in the left hand part of graph 515 in FIG. 5) will result in step changes, discontinuities or other irregularities in the Motion Phase acceleration waveform, a lower peak velocity, and a slower overall Motion Phase. These are sometimes referred to as the “Motor Task Indicators” as explained below.

Motor Task Indicators consist of a time-dependent acceleration or velocity waveform. The waveform is representative of the Interaction Phase of the Motor Task. The Interaction Phase is shown in the “Interaction Phase” of graph 505 in FIG. 5. The Interaction Phase is associated with the part of the Motor Task where the individual performs some interaction with the object, for example:
A hand grabbing motion (e.g. when grabbing a cup).
A hand press-and-release motion (e.g. when releasing earphone after placing in ear).
A pointed finger pushing motion (e.g. pushing a button).

The Interaction Phase of the Motor Task has a particular time-dependent reference acceleration or velocity waveform associated with it. In other words, when an individual grabs a cup, the movement of the individual during this phase will produce a particular time-dependent acceleration or velocity waveform that is unique to that particular action. This unique time-dependent acceleration or velocity waveform will be produced irrespective of the individual performing the action. This means that a reference time-dependent acceleration or velocity waveform of known Motor Tasks can be produced for the individual under test to be compared against to allow the particular Motor Task carried out by the individual under test to be identified. These reference time-dependent acceleration or velocity waveforms are stored in the Motor Task Indicator Database. The reference motion data is, in embodiments, captured from known healthy individuals. In other words, the reference motion data is captured from individuals with no or little cognitive impairment. In embodiments, the reference motion data is captured over a period of time that includes a plurality of motor tasks.

In embodiments, the reference motion data is captured from the individual themselves and is thus used to determine any changes to cognitive impairment over a period of time. In this case, the reference motion data is captured from the individual over a time that is longer than the time period for one motion task.

The Motor Task Indicator Database may be populated prior to system use with Motor Task Indicators recorded from motion data captured from the IMU device 300. Motor Task Indicators may be generic, average or idealised waveforms derived from the motion data of many individuals. These individuals may be from a cohort similar to the individual under test. For example, these individuals may be of a similar age or have a similar demographic profile to the individual under test. Of course, the disclosure is not so limited and may be recorded for a specific individual.

Motor Task Indicators may have an associated identification number or string (the “Motor Task Indicator ID”). These uniquely identify one reference Motor Task from another Motor Task. Accordingly, the Motor Task Indicator ID should be capable of doing this.

In embodiments, Motor Task Indicators may additionally include signatures related to other data types (aside from motion data). Such signatures may be sensed by other systems to augment Motor Task identification. In other words, in addition to or instead of the time-dependent acceleration or velocity waveforms, other reference motion data is envisaged. In embodiments, this include, for example: Pressure pattern signatures; Myoelectric signatures (e.g. to be sensed by worn patches); Device-specific actions (e.g. signature of hinge sensors opening on an earphone case); Acoustic signatures related to specific actions (e.g. turning key, opening door) or the like.

The Motor Task Indicator Identification Algorithm identifies within motion data any acceleration or velocity waveform patterns that match known Motor Task Indicators retrieved from the Motor Task Indicator Database. In other words, the motion data captured from the individual is compared to the stored reference time-dependent acceleration or velocity waveform of Interaction Phases of known Motor Tasks. Where there is a positive comparison, the Motor Task Indicator is identified. Accordingly, the algorithm outputs: a list of timestamps indicating when in the motion data Motor Task Indicators were identified. These will be referred to as the Identified Motor Task Indicators. For each timestamp, the associated Motor Task Indicator ID as retrieved from the Motor Task Indicator Database.

The IMU Data Segmentation Algorithm segments the motion data based on the Identified Motor Task Indicator timestamps, outputting the relevant motion data segments. These relevant motion data segments are referred to as the “Segmented IMU Data” and are output individually. The Segmented IMU Data may consist of an acceleration or velocity waveform including the data which occurs immediately prior to an Identified Motor Task Indicator. Note that this waveform should represent the Motion Phase of the Motor Task. In other words, as the Interaction Phase of the Motor Task is identified from the comparison of the Motion Data of the individual under test with the stored reference time-dependent acceleration or velocity waveform of known Motor Tasks, the Motion Phase of the individual performing that particular Motor Task can be identified. The Motion Phase occurs during the first time period.

In embodiments, the Segmented IMU Data also inherits the Motor Task Indicator ID of its associated Identified Motor Task Indicator. Each instance of Segmented IMU Data is also assigned a unique identification string or number which is referred to as the “Segment ID”.

The IMU Data Motion Assessment Algorithm assesses Segmented IMU Data for Motion Phase properties of relevance for later determining MCI risk (the “Motion Assessment Data”). In embodiments, the Motion Assessment Data consists of: the total length in time of the IMU Data segment (i.e. the total length in time of the Motion Phase) (the “Motion Phase Duration”); the absolute value of the maximal velocity over the IMU Data segment (the “Maximal Velocity”); the difference in time between the moment of maximal velocity and the midpoint of the IMU Data segment (the “Midpoint Error”). It will be appreciated that the correlation between the Motion Assessment Data and the MCI Risk is determined from research identified in Non Patent Literature 1, the contents of which is incorporated herein by reference.

The Motion Assessment Data also inherits the Segment ID and the Motor Task Indicator ID. The Motion Assessment Database stores the Motion Assessment Data with the associated Segment ID and Motor Task Indicator ID. The MCI Risk Assessment Algorithm uses the Motion Assessment Data, and in optional embodiments data from other sources (the “Other MCI Relevant Data”) to calculate an MCI risk score (the “MCI Risk”). As noted above, the MCI Risk is calculated using the research carried out in Non Patent Literature 1. The MCI Risk may be a numerical score representing a calculated risk factor that the tested individual has MCI. In embodiments, the MCI Risk may be calculated for an individual based on an average across many instances of collected Motion Assessment Data. For example, the MCI Risk may be calculated across many different types of Motor Task such as gripping a cup for using a key in a lock. However, the disclosure is not so limited. In other instanced, the MCI Risk may be calculated using one specific Motor Task type, as indicated by the Motor Task Indicator ID. In other words, the MCI Risk may be attributed to the performance of only one task. The MCI Risk calculation may be refined in embodiments using other MCI Relevant Data. The other MCI Relevant Data may be speech data, eye tracking data or other bodily motion data and is primarily used to refine the MCI Risk calculation and rule out other disorders with similar motor symptoms of MCI.

In the MCI RN device 200, various algorithms are carried out by the MCI RN processing circuitry 210. These are the notification algorithm and the alert algorithm. The computer program code used to carry out these algorithms is stored in the MCI RN storage 220. One of these algorithms will be described now.

The Notification Algorithm calculates the priority of an MCI Notification (the “MCI Notification Priority”), based on the MCI Risk. The MCI Notification Priority in embodiments details the type of MCI Notification. In other words, whether the notification is an audio or visual (or both) notification. In embodiments the MCI Notification Priority details the invasiveness of the MCI Notification for example the volume of an audio notification. Further, in embodiments, the MCI Notification Priority details the timing of the MCI Notification. For example, the MCI Notification may wake the individual or their medical practitioner if the MCI Risk is very high.

As already noted, the IMU device 300 is provided to capture motion data of the individual and to pass the captured motion data to one or both of the MCI RA device 100 and/or the MCI RN device 200. In embodiments, one or more the above noted algorithms may be performed on the IMU device 300 and/or one or more of the above noted databases may be stored on the IMU device 300. In other words, although the foregoing describes one algorithm as being performed in one device and one database being stored on one particular device, the disclosure is not so limited and algorithms and/or databases may be provided in any one device.

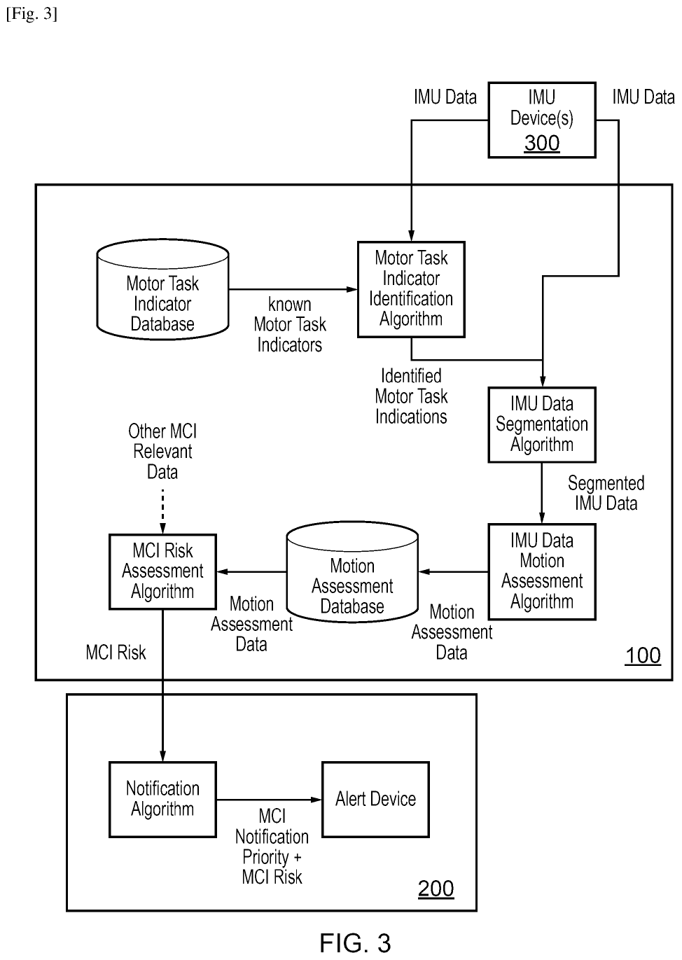

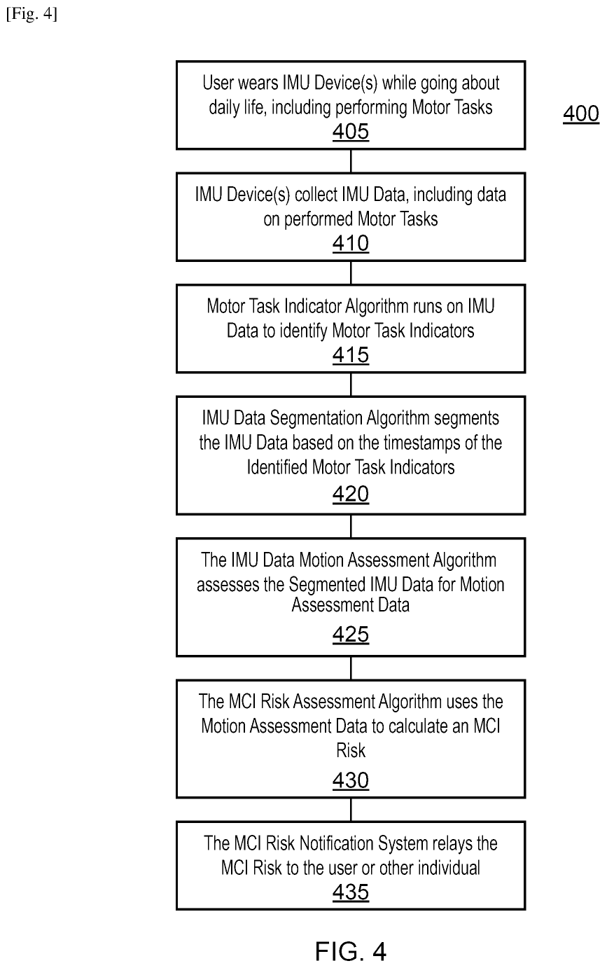

FIG. 3 shows the functional diagram of FIG. 2 in more detail. In particular, the embodiments of FIG. 3 show the interrelationship between each of the above noted algorithms in the system of FIG. 2. This will become apparent when the process according to embodiments of the disclosure is described in relation to FIG. 4.

FIG. 4 shows a process according to embodiments of the disclosure.

The process 400 starts and in step 405 the individual wears/uses one or more IMU Devices while going about their daily life. In embodiments, the IMU Device 300 is worn continuously in a body location to gather motion data on Motor Tasks. For example, the IMU device 300 is worn on the wrist (e.g. embodied as a mSafety watch) or on the finger (e.g. embodied as a smart ring). However, the disclosure is not so limited an in other embodiments, some IMU Devices 300 may be able to provide periodic information on Motor Tasks, such as headphones or hearing aids being picked up and placed in the ear, or smartphones held in the hand.

The individual naturally performs Motor Tasks as part of their daily life. Motor Tasks may include: moving the arm and hand to grasp an object, such as a cup; moving a hand to interact with an object with a pointed finger, such as interacting with a smartphone or tablet; or picking up or placing wearable devices on the body, such as earphones, head mounted displays, etc.

The process moves to step 410. In step 410, the IMU Device 300 collects IMU data, including data on performed Motor Tasks. In embodiments, the IMU Device 300 collects IMU data continuously, such as those worn on the wrist or finger. However, in embodiments, the IMU Device 300 will collect useful IMU Data associated with specific events. This may be of particular relevance for earphone/hearing aid embodiments, where, for example, IMU Data collection may be triggered by the individual touching the device when picking it up, and ends when the device is released after placing it in the ear. In embodiments IMU Devices 300 may only gather partial IMU Data on any given Motor Task. For example, in the event of the Motor Task being the individual listening to music, one IMU device 300 will be a smartphone and a second IMU device 300 will be the individual's headphones. In this case, the smartphone may only be able to identify where the individual grips the smartphone and activate the music app, but the headphones will be required to provide IMU data of the individual placing the headphones in their cars. In other words, in some instance, the IMU Data of multiple IMU devices 300 may be required.

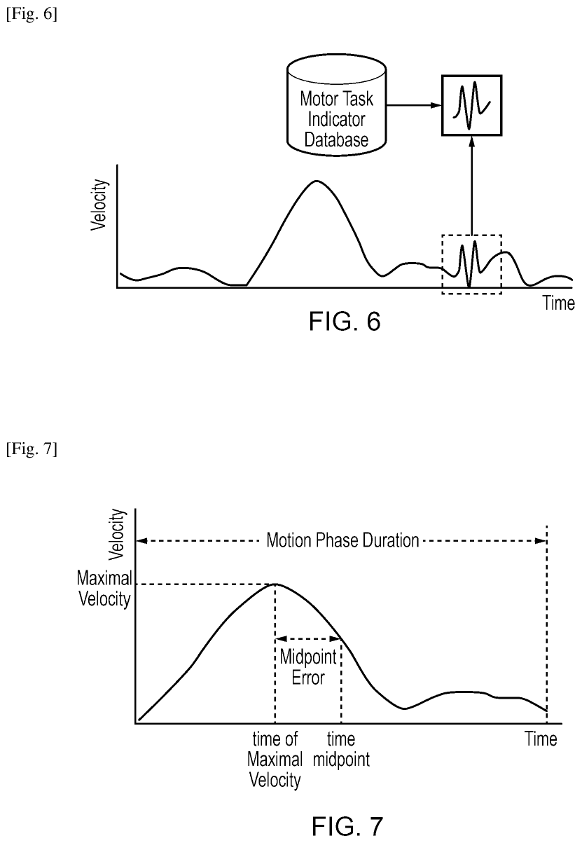

The process moves to step 415. In step 415, the Motor Task Indicator Algorithm runs on the IMU Data to identify Motor Task Indicators, outputting the Identified Motor Task Indicators. FIG. 6 shows one method for accomplishing this according to embodiments.

Referring to FIG. 6, for each known Motor Task Indicator retrieved from the Motor Task Indicator Database the Motor Task Indicator algorithm performs a search in the IMU Data to identify segments of the waveform that match the Motor Task Indicator waveform. A match is made when the difference between the waveform and the Motor Task Indicator waveform is below a predetermined threshold. In order to achieve this, the Motor Task Indicator algorithm steps through the IMU Data in small time intervals (e.g. 0.2 s), considering a small segment (e.g. 1 to 3 s) of the IMU data at a time. At each step, the Motor Task Indicator algorithm compares the time-amplitude relationship of the considered Motor Task Indicator waveform to the waveform in the IMU Data segment. If the average amplitudinal and temporal differences between the Motor Task Indicator waveform and IMU Data segment waveform are below the predetermined threshold, then the IMU Data segment is identified as matching the Motor Task Indicator.

In embodiments, the above steps are repeated multiple times with different scaling applied to the time axis of the Motor Task Indicator, i.e. the Motor Task Indicator may be scaled in the time axis to account for faster or slower Interaction Phases.

Once identified, the timestamp of the onset of the identified Motor Task Indicator is retained, as well as the corresponding Motor Task Indicator ID.

In embodiments, additional non-IMU Data Motor Task Indicators are also to be used. In this case, similar methods to the search method noted above may be utilised for their specific data type to identify the timestamp of the onset of the Motor Task Indicator. For example, for a “earphone case opening” Motor Task Indicator, the timestamp may be defined as the moment the case opens.

Once the steps of the search method have been completed for every Motor Task Indicator retrieved from the Motor Task Indicator Database, the algorithm outputs the list of timestamps and corresponding Motor Task Indicator IDs as the Identified Motor Task Indicators.

Returning to FIG. 4, the process continues at step 420. In step 420, the IMU Data Segmentation Algorithm segments IMU Data based on the Identified Motor Task Indicators and outputs the Segmented IMU Data. In embodiments, this is achieved by: For each Identified Motor Task Indicator: starting at the corresponding Identified Motor Task Indicator timestamp, the IMU Data Segmentation algorithm searches backwards through time on the IMU Data waveform to identify the initial start of the Motion Phase of the Motor Task, i.e. the point in time at which acceleration and rate of change of acceleration are 0. The IMU Data Segmentation algorithm then segments the IMU data, using this determined timestamp as the start of the waveform segment and the Identified Motor Task Indicator timestamp as the end of the waveform segment. The IMU Data segments are assigned a unique Segment ID, as well as inheriting the Motor Task Indicator ID of their corresponding Identified Motor Task Indicator.

The IMU Data segments and their associated IDs are output as the Segmented IMU Data.

The process moves to step 425 where the IMU Data Motion Assessment Algorithm assesses the Segmented IMU Data to output the Motion Assessment Data. The goal of the IMU Data Motion Assessment Algorithm is to extract and/or calculate Motion Phase properties from the Segmented IMU Data which may later be utilised in the MCI Risk calculation.

For each segment of the Segmented IMU Data, properties of the Motion Phase are determined or calculated. This is shown with reference to FIG. 7. Specifically, the Motion Phase Duration is, in embodiments, calculated by subtracting the IMU Data segment's start timestamp from its end timestamp. The Maximal Velocity is extracted by identifying the largest amplitude value for the entire waveform of the IMU Data segment.

In embodiments, the Midpoint Error is calculated by: identifying the midpoint between the IMU data segment's start timestamp and end timestamp; and calculating the difference between the midpoint and the time value corresponding to the point of Maximal Velocity.

The extracted values for a segment are stored in a table as the Motion Assessment Data, along with their corresponding Motor Task Indicator ID and Segment ID.

The Motion Assessment Data is timestamped and stored in the Motion Assessment Database.

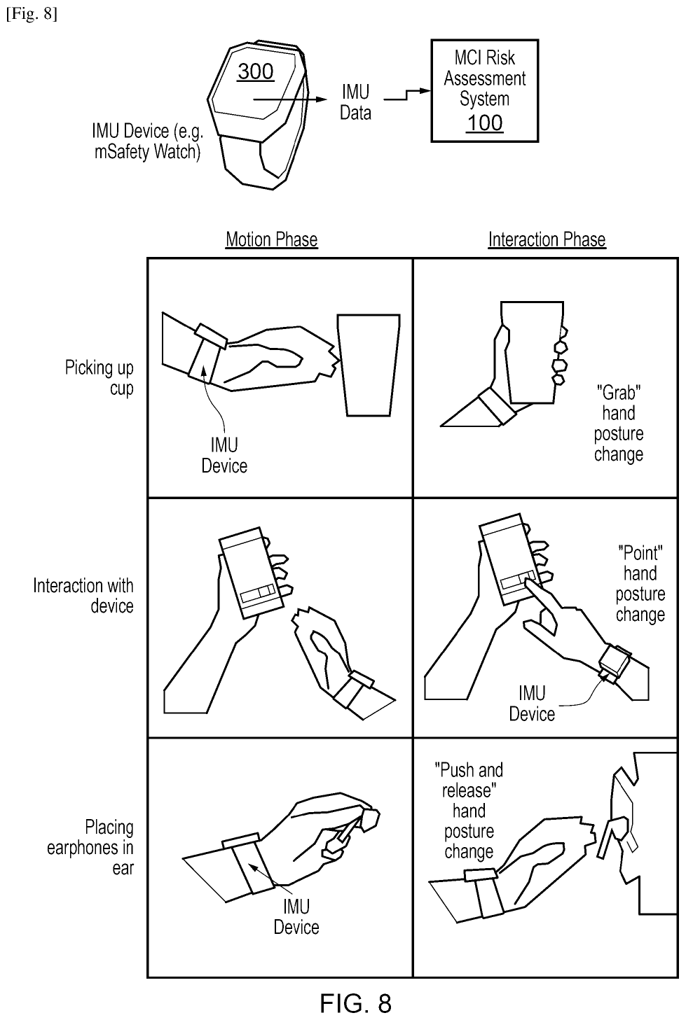

In FIG. 8, several typical movements are shown. These are broken down into the motion phase (during the first time period) and the interaction phase (during the second time period). In the top Motor Task, an individual is picking up a cup. The motion phase has the individual moving their hand towards the cup. This is left hand part of the graphs in FIG. 4. The individual then grabs the cup in the interaction phase. This is the right hand part of the graphs of FIG. 5. As noted above, the motion data of this entire movement is captured by the IMU device 300 and passed to the MCI RA device 100. The MCI device 100 compares the captured motion data with stored interaction phase data and where there is a match (in this case the IMU data of the grabbing motion) the motion data preceding the interaction phase data is compared with stored reference motion data for the grabbing of a cup and the MCI Risk is calculated.

In the middle Motor Task, an individual interacting with a smartphone is shown. In this case, the motion phase has the individual moving their hand towards a smartphone. The interaction phase is therefore a point hand gesture. Finally, in the bottom Motor Task, an individual placing earphones in their ear is shown. In this case, the motion phase has the individual moving their hand towards their ear. The interaction phase is the individual putting the earphone in their ear and releasing their hand.

In embodiments the motion data may be captured continuously over the entire motion phase. However, in embodiments, the motion data may be captured periodically over the time period and the indicator value (which itself is used to determine the cognitive impairment) is determined using the periodically captured motion data. In embodiments, the time period over which the motion data is captured may include one or more motion phases. For example, the time period over which the motion data is periodically captured may be a day, a week or longer. This would include a plurality of motion phases and would provide more information relating to the gradual change in cognitive impairment.

Returning to FIG. 4, the process moves to step 430. In step 430, the MCI Risk Assessment Algorithm uses the Motion Assessment Data, and in embodiments Other MCI Relevant Data, to calculate an MCI Risk.

The MCI Risk Assessment Algorithm may be triggered once an acceptable amount of Motion Assessment Data has been collected. In embodiments, the quality of the MCI Risk value scales with the amount of Motion Assessment Data. Thus the longer the time the user has had their Motor Tasks monitored, the more accurate the calculated MCI Risk will be. Moreover, as Motor Tasks are a very common class of human motion, hundreds of Motor Tasks should be available for monitoring each day. Therefore, in embodiments, an acceptable amount of Motion Assessment Data for an initial MCI Risk calculation should be available within a week to a month of initial monitoring.

In embodiments, the precise value for an acceptable amount of Motion Assessment Data entries is investigated experimentally and compared to accepted diagnosis criteria for MCI for a more precise required data collection duration. In other words, an acceptable level of accuracy of MCI is determined and then the required data collection duration is determined experimentally. In embodiments, therefore, the amount of movement an individual during an average day will determine the required data collection duration.

In embodiments, global averages for healthy individuals may be used as a baseline comparison for Motion Assessment Data, against which the MCI Risk may be determined. Equivalent Motion Assessment Data on healthy individuals (ideally within the same demographic as the individual being tested, such as age, gender, fitness level, etc.) may have been collected from peer-reviewed research, or collected by this system and method on individuals who are known to be healthy.

In embodiments, the tested individual's Motion Assessment Data is first averaged for each Motor Task type. So, for each Motor Task type the MCI Risk Assessment Algorithm uses the Motor Task Indicator IDs to retrieve all instances of Motion Assessment Data for the given Motor Task type. The values present in the Motion Assessment Data are then averaged. Accordingly, each Motor Task type has an associated average set of Motion Assessment Data.

For each Motor Task type, the Motion Assessment Data of the tested individual is compared to the average values of the healthy cohort. For example, a lower Maximal Velocity compared to the healthy cohort indicates an increase in MCI Risk. Advanced MCI individuals are expected to have around a 40% decrease in maximal velocity compared to healthy individuals of the same demographic. An increase in the Motion Phase Duration compared to the healthy cohort indicates an increase in MCI Risk. Advanced MCI individuals may be expected to have around twice as long Motion Phase Duration compared to healthy individuals of the same demographic as the individual. An increase in the Midpoint Error compared to healthy cohort. Advanced MCI individuals are also expected to have Midpoint Errors around 500% larger compared to healthy individuals in the same demographic.

In embodiments, a final MCI Risk score is calculated based on the size of the differences between the tested individual's Motor Assessment Data and the healthy cohort's data for each Motor Task type. In embodiments, different Motor Task types may be weighted differently in the final calculation. For example, Motor Tasks involving large limb motions may be weighted more highly than those involving fine finger motions, as they are more useful for diagnosing cognitive impairment over other disorders such as arthritis.

In embodiments, successively acquired Motion Assessment Data may be compared for the individual under test, such that the change in their Motor Planning Error may be used as part of the MCI Risk calculation. This monitors whether an individual's MCI performance is decreasing over time. This enables the performance of medication or other therapy to be monitored.

In order to do this, for each Motor Task Indicator ID present in the Motion Assessment Database, the algorithm retrieves all corresponding Motion Assessment Data and timestamps. The change in Motion Assessment Data over time is assessed. For example, a decrease in Maximal Velocity over time indicates in an increase in MCI Risk. Further, an increase in the total length of the Motion Phase indicates an increase in MCI Risk and an increase in the Midpoint Error over time indicates an increase in MCI Risk.

In embodiments, the algorithm uses the rate of change of Motion Assessment Data values over time, as well as optionally demographic information about the individual such as age, to compare symptom progression curves to known standard progression curves of MCI symptoms of different severities, and therefore output a numerical MCI Risk. This will be provided to the MCI RN device 200 as will be explained later.

In embodiments, the MCI Risk Assessment algorithm performs further operations to better differentiate the MCI Risk from risk factors of other diseases or disorders. The methods for accomplishing this may vary depending on the category of other disease.

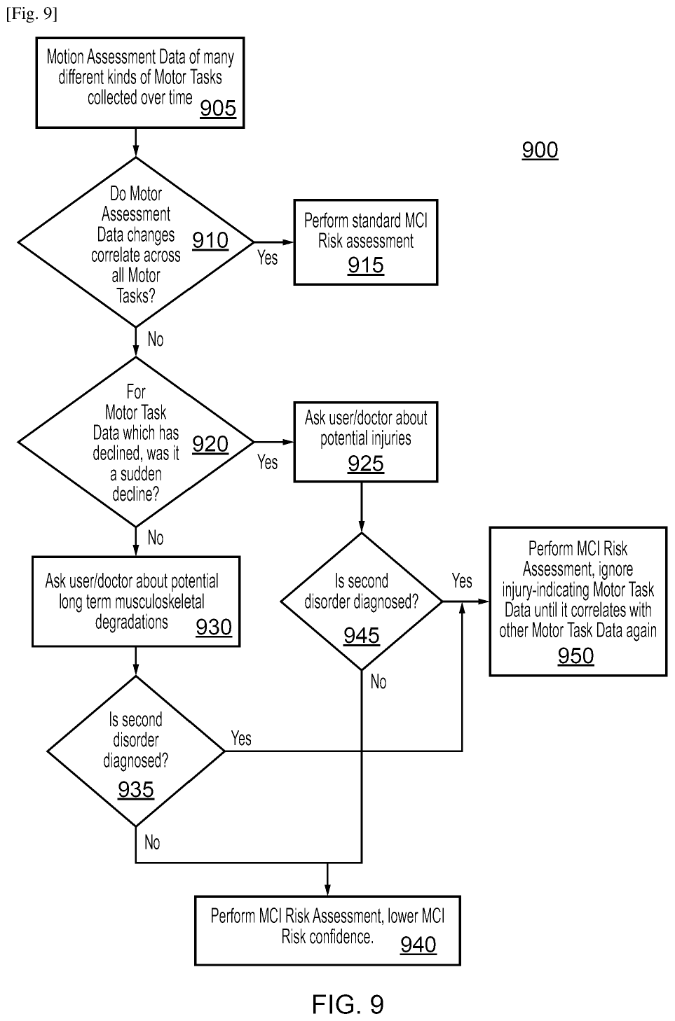

In embodiments, for differentiating MCI Risk from musculoskeletal issues such as injury, changes in muscle strength, arthritis or the like, the MCI Risk Assessment algorithm may compare the Motion Assessment Data of different Motor Tasks to each other over time. This will be explained later with reference to FIG. 9. If changes in Motion Assessment Data correlate across all Motor Tasks, such changes may be reliably attributed to a change in motor cognitive ability, and thus may be used in the MCI Risk calculation. However, if changes in the Motion Assessment Data are only present in certain Motor Tasks (e.g. only present for Motor Tasks with a “pinching” Motor Task Indicator ID), then such changes may be more indicative of a musculoskeletal problem, and the MCI Risk may be lowered or unaltered.

In embodiments, if there is low confidence, the algorithm may trigger the MCI Risk Notification System to prompt the user to ask directly if there is an injury in a particular area (e.g. “Have you injured your hand?”).

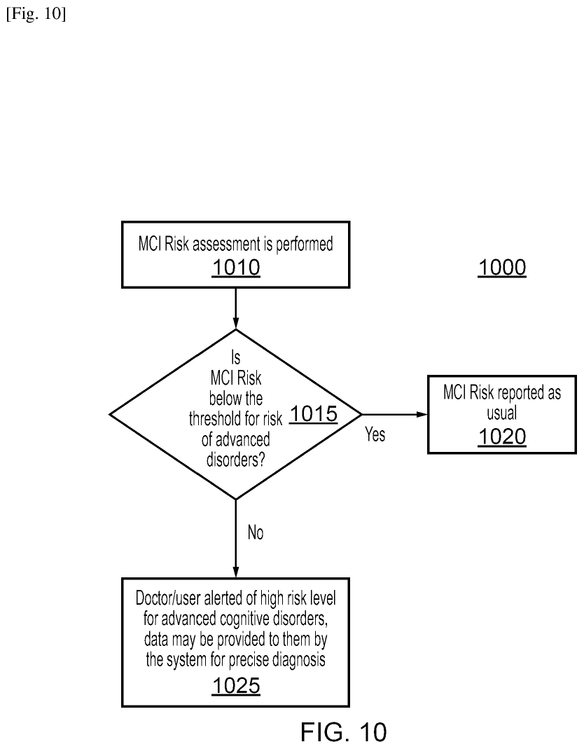

In embodiments, the MCI Risk Assessment algorithm differentiates MCI Risk from advanced cognitive disorders, such as Parkinson's disorder, Alzheimer's disorder, other dementia disorders or the like. This will be described with reference to FIG. 10. However, MCI symptoms broadly present milder versions of the same symptoms present in more advanced cognitive disorders, and MCI is considered a key predictor of developing these disorders. Therefore, MCI Risk is itself used as the risk factor for such disorders in embodiments. Once MCI Risk has been calculated for an individual, it is compared to a pre-established scale for cognitive disorder risks. If the MCI Risk is beyond a threshold, the MCI Risk Notification System is triggered to immediately notify the individual or their doctor that they need to be checked for more advanced cognitive disorders such as Parkinson's or Alzheimer's. Broadly, as the system and method described here are primarily focused on detecting small discrepancies in Motor Planning Error for identifying MCI, very large discrepancies are easy to identify.

In embodiments, Other MCI Relevant Data may be used to refine the MCI diagnosis to rule out other disorders. This Other MCI Relevant Data is physiological data captured by a second wearable device worn by the individual. The type of Other MCI Relevant Data utilised may vary depending on the disorders to be differentiated. For example, in embodiments, saccadic eye movement is determined from eye tracking data. These may be used to predict Parkinson's disorder. Eye tracking data may be collected on the individual in addition to IMU Data, such that a differential diagnosis may be made. If there are strong Parkinson's indicating symptoms present in the saccadic eye movement data in addition to Motor Planning Error symptoms in the IMU Data, the user may be assigned a “Parkinson's Risk” tag in addition to their numerical MCI Risk score. If there are no Parkinson's-indicating symptoms present in the eye movement data, then the individual may be assigned an MCI Risk score as usual.

FIG. 9 shows a process 900 explaining Musculoskeletal Disorder Differentiation. The process starts and progresses to step 905 where Motion Assessment Data of many different kinds of Motor Tasks are collected over a period of time. In embodiments, different kinds of Motor Tasks may include those shown in FIG. 8 such as grabbing a cup, interacting with a smartphone or placing headphones in the individual's cars. Other Motor Tasks may include inserting a key into a lock and opening a door, writing a word or the like. In embodiments, the different kinds of Motor Tasks will involve different muscle groups. This will help better differentiate between MCI and other musculoskeletal issues which tend to affect the same or similar muscle groups. The Motion Assessment Data over the different kinds of tasks may be captured using one or more different wearable devices. For example, the Motion Assessment Data for inserting a key into a lock or opening a door may be captured from a smart watch or ring or the like.

The process then moves to step 910 where the Motor Assessment Data changes across all Motor Tasks are compared. This is useful because if only certain motor actions are affected rather than all motor actions being affected, the reason for the decline is more likely caused by a particular injury or second disorder rather than being a broader cognitive issue. In the event that the changes do correlate such that the changes occur across a broader range of motor issues, the “yes” path is followed to step 915 where an MCI Risk Assessment is carried out. In other words, in the event that the correlation between the changes in the Motor Tasks is above a predetermined threshold, the “yes” path is followed.

However, in the event that the changes do not correlate (and so a smaller number or range of motor actions are affected), the “no” path is followed to step 920. In step 920, the amount of the decline in the Motor Task is obtained. This may be the amount of decline since the last test or the amount of decline in a particular given period of time. By analysing the amount of decline, it is possible to determine if the decline is sudden. If it is determined that the decline is a sudden decline, the “yes” path is followed to step 925. However, if the decline is not a sudden decline, then the process moves to step 930.

In step 925, the individual and/or medical practitioner is asked to confirm if the individual has suffered any injuries which would explain the sudden decline. The process then moves to step 945.

In step 945 a second disorder may be diagnosed. The second disorder may be diagnosed by either the medical practitioner or the individual. In the case of the individual diagnosing the second disorder, the medical practitioner may be asked to confirm the diagnosis. For example, the user may be allowed to enter that they have injured their hand and the medical practitioner can confirm the extent of the injury. In other example embodiments, the user diagnosed injury may be confirmed by an automated system which asks the individual to capture an image or video of the affected area. In embodiments, the severity of the second disorder may be noted. This will quantify the impact of the second disorder on the MCI Risk Assessment.

If a second disorder is diagnosed, the “yes” path is followed to step 950 where the MCI risk assessment is carried out. In this instance, the disorder, and in embodiments the severity of the disorder, is used to mitigate the impact of the second disorder on the MCI Risk Assessment. In other words, the MCI Risk Assessment is performed which ignores the impact of the second disorder.

In embodiments, the second disorder may be selected from a predefined list of diagnoses and associated motion phase characteristics. As there are motion characteristics associated with the diagnoses, it is possible for the MCI Risk Assessment to be performed ignoring the impact of the second disorder. In embodiments, it is also envisaged that the Motion Phase data of Motor Tasks associated with the second disorder is not included in any calculation of the MCI Risk Assessment. In other words, the Motor Assessment changes that do not correlate across all Motor Tasks are not included.

However, in the event that a second disorder is not diagnosed, the “no” path is followed to step 940.

Returning to step 930, if the decline was not sudden, the individual and/or the medical practitioner is asked about potential long term musculoskeletal degradations. This may be provided as a list of questions such as “has the individual found it increasingly difficult to pick up objects over the past six months”. This will provide an indication if the degradations have occurred over a long period to reduce the likelihood of an injury causing the degradation.

The process then moves to step 935 where a second disorder is checked in a similar manner to step 945 as explained above. Accordingly, for brevity, this will not be explained hereinafter. If a second disorder is diagnosed, the “yes” path is followed to step 950 as explained above. However, if a second disorder is not diagnosed, the “no” path is followed to step 940.

In step 940, the MCI Risk Assessment is performed. However, in this case, the MCI risk confidence is lowered. This is because a second disorder is not identified by the system. Accordingly, in the event that the MCI risk confidence is below a threshold, the individual and/or the medical practitioner is asked if the individual has been injured or is suffering an injury.

FIG. 10 shows a process 1000 explaining Advanced Cognitive Disorder Differentiation. The process starts and moves to step 1010 where an MCI Risk assessment is performed as explained above. The process moves to step 1015 where a check is made to see if the MCI Risk is below a threshold for advanced cognitive disorders such as Parkinson's disorder. This threshold is determined by carrying out the MCI Risk assessment on known suffers of these advanced cognitive disorders to determine the appropriate MCI Risk Assessment value for such disorders. If the MCI Risk for the individual is below the threshold, the “yes” path is followed to step 1020 where the MCI Risk is reported by the MCI RN device 200 as normal. If, however, the MCI Risk for the individual is above the threshold, the “no” path is followed to step 1025. In this case, the individual and/or medical practitioner is notified of the high risk level so that the individual may be re-tested or tested for the advanced cognitive disorder. In embodiments, information relating to the MCI Risk Assessment may be provided. For example, the muscle group or the particular Motor Task that raised the MCI Risk in the individual may be provided as part of the notification process. This will assist in the diagnosis of any advanced cognitive disorder.

Although the foregoing has been described with the Interaction Phase of the captured Motor Task being used to automatically determine the Motor Task being carried out by the individual, the disclosure is not so limited. In embodiments, the individual interacts with a particular object, such as a smartphone. In these embodiments, the object may communicate with one or both of the IMU device 300 or the MCI RA device 100 to identify the interaction of the individual. Additionally, the position of various objects (such as the position of a smart-lock) may be pre-defined and then the IMU device 300 may provide its location to the MCI RA device 100. Accordingly, if the IMU device 300 is in proximity to the smart-lock, the MCI RA device 100 will, in embodiments, compare the captured Motion Data to the interactions with a smart-lock first for comparison as it is more likely that the Motor Task is associated with the interaction with the smart-lock.

Returning to FIG. 4, the process finally moves to step 435. In step 435, the MCI RN device 200 provides the calculated MCI Risk to the individual or a medical practitioner. In order to determine the notification method, a notification priority needs to be determined. The notification priority may be calculated based on either The size of the calculated MCI Risk (i.e. higher priority if MCI Risk is large); or the size of the change between latest MCI Risk calculation and the previous (i.e. higher priority if there are sudden changes). In other words, the notification priority defines the urgency that needs to be given to the notification when communicating this to the individual or the medical practitioner.

The MCI RN device 200 displays the MCI Notification according to the MCI Notification Priority. The MCI RN device 200 may take pre-defined actions depending on the size of the MCI Notification Priority, such as if the MCI Notification Priority is low, the MCI RN device 200 may simply create an indicator in the device's notification list that the MCI Notification is available to view whenever convenient or where a Notification Priority threshold has been exceeded, a pop-up notification containing the MCI Notification appears, or a notification sound, vibration or alarm is sounded. Further, the individual's medical practitioner may also be alerted either in the event of a very high MCI Risk or whenever an MCI Risk score is provided to the individual.

The MCI Notification may be displayed on the MCI RN device 200 as a single MCI Risk figure, but may also include graphs indicating change of MCI Risk over time; details of change in Motion Assessment Data of different Motor Task types over time or the like. Additionally, a viewer may view a “picking up cup” Motor Task and sec the change in performance of that task versus a “pressing button” Motor Task.

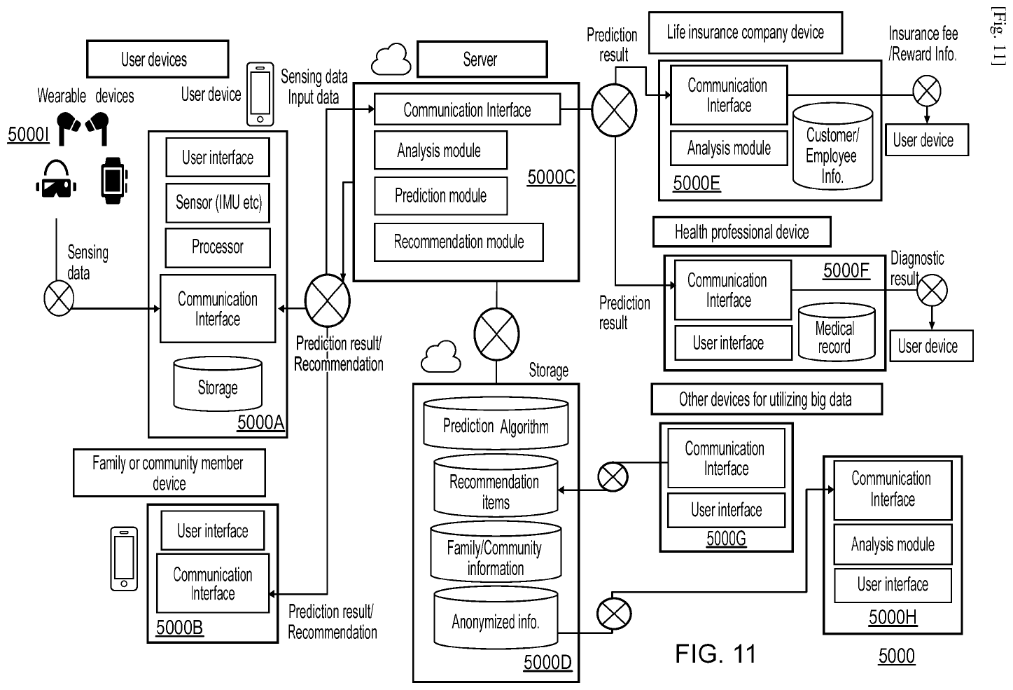

Although the foregoing has been described with reference to embodiments being carried out on a device or various devices, the disclosure is not so limited. In embodiments, the disclosure may be carried out on a system 5000 such as that shown in FIG. 11.

In the system 5000, the wearable devices 5000I are devices that are worn on a user's body. For example, the wearable devices may be earphones, a smart watch, Virtual Reality Headset or the like. The wearable devices contain sensors that measure the movement of the user and which create sensing data to define the movement or position of the user. This sensing data is provided over a wired or wireless connection to a user device 5000A. Of course, the disclosure is not so limited. In embodiments, the sensing data may be provided directly over an internet connection to a remote device such as a server 5000C located on the cloud. In further embodiments, the sensing data may be provided to the user device 5000A and the user device 5000A may provide this sensing data to the server 5000C after processing the sensing data. In the embodiments shown in FIG. 11, the sensing data is provided to a communication interface within the user device 5000A. The communication interface may communicate with the wearable device(s) using a wireless protocol such as low power Bluetooth or WiFi or the like.

The user device 5000A is, in embodiments, a mobile phone or tablet computer. The user device 5000A has a user interface which displays information and icons to the user. Within the user device 5000A are various sensors such as gyroscopes and accelerometers that measure the position and movement of a user. The operation of the user device 5000A is controlled by a processor which itself is controlled by computer software that is stored on storage. Other user specific information such as profile information is stored within the storage for use within the user device 5000A. As noted above, the user device 5000A also includes a communication interface that is configured to, in embodiments, communicate with the wearable devices. Moreover, the communication interface is configured to communicate with the server 5000C over a network such as the Internet. In embodiments, the user device 5000A is also configured to communicate with a further device 5000B. This further device 5000B may be owned or operated by a family member or a community member such as a carer for the user or a medical practitioner or the like. This is especially the case where the user device 5000A is configured to provide a prediction result and/or recommendation for the user. The disclosure is not so limited and in embodiments, the prediction result and/or recommendation for the user may be provided by the server 5000C.

The further device 5000B has a user interface that allows the family member or the community member to view the information or icons. In embodiments, this user interface may provide information relating to the user of the user device 5000B such as diagnosis, recommendation information or a prediction result for the user. This information relating to the user of the user device 5000B is provided to the further device 5000B via the communication interface and is provided in embodiments from the server 5000C or the user device 5000A or a combination of the server 5000C and the user device 5000A.

The user device 5000A and/or the further device 5000B are connected to the server 5000C. In particular, the user device 5000A and/or the further device 5000B are connected to a communication interface within the server 5000C. The sensing data provided from the wearable devices and or the user device 5000A are provided to the server 5000C. Other input data such as user information or demographic data is also provided to the server 5000C. The sensing data is, in embodiments, provided to an analysis module which analyses the sensing data and/or the input data. This analysed sensing data is provided to a prediction module that predicts the likelihood of the user of the user device having a condition now or in the future and in some instances, the severity of the condition. The predicted likelihood is provided to a recommendation module that provides a recommendation to the user and/or the family or community member. Although the prediction module is described as providing the predicted likelihood to the recommendation module, the disclosure is not so limited and the predicted likelihood may be provided directly to the user device 5000A and/or the further device 5000B.

Additionally, connected to or in communication with the server 5000C is storage 5000D. The storage 5000D provides the prediction algorithm that is used by the prediction module within the server 5000C to generate the predicted likelihood. Moreover, the storage 5000D includes recommendation items that are used by the recommendation module to generate the recommendation to the user. The storage 5000D also includes in embodiments family and/or community information. The family and/or community information provides information pertaining to the family and/or community member such as contact information for the further device 5000B.

Also provided in the storage 5000D is an anonymised information algorithm that anonymises the sensing data. This ensures that any sensitive data associated with the user of the user device 5000A is anonymised for security. The anonymised sensing data is provided to one or more other devices which is exemplified in FIG. 11 by device 5000H. This anonymised data is sent to the other device 5000H via a communication interface located within the other device 5000H. The anonymised data is analysed with the other data 5000H by an analysis module to determine any patterns from a large number set of sensing data. This analysis will improve the recommendations made by the recommendations module and will improve the predictions made from the sensing data. Similarly, a second other device 5000G is provided that communicates with the storage 5000D using a communication interface.

Returning now to server 5000C, as noted above, the prediction result and/or the recommendation generated by the server 5000C is sent to the user device 5000A and/or the further device 5000B.

Although the prediction result is used in embodiments to assist the user or his or her family member or community member, the prediction result may be also used to provide more accurate health assessments for the user. This will assist in purchasing products such as life or health insurance or will assist a health professional. This will now be explained.

The prediction result generated by server 5000C is sent to the life insurance company device 5000E and/or a health professional device 5000F. The prediction result is passed to a communication interface provided in the life insurance company device 5000E and/or a communication interface provided in the health professional device 5000F. In the event that the prediction result is sent to the life insurance company device 5000E, an analysis module is used in conjunction with the customer information such as demographic information to establish an appropriate premium for the user. In instances, rather than a life insurance company, the device 5000E could be a company's human resources department and the prediction result may be used to assess the health of the employee. In this case, the analysis module may be used to provide a reward to the employee if they achieve certain health parameters. For example, if the user has a lower prediction of ill health, they may receive a financial bonus. This reward incentivises healthy living. Information relating to the insurance premium or the reward is passed to the user device.

In the event that the prediction result is passed to the health professional device 5000F, a communication interface within the health professional device 5000F receives the prediction result. The prediction result is compared with the medical record of the user stored within the health professional device 5000F and a diagnostic result is generated. The diagnostic result provides the user with a diagnosis of a medical condition determined based on the user's medical record and the diagnostic result is sent to the user device.

In the context of embodiments of the disclosure, the MCI RA 100 is either the user device 5000A or the server 5000C as this receives information about the movement and/or position of the user from wearable devices 5000I. Moreover, the wearable devices 5000I that generate the sensing data are, in embodiments, the IMU device 300 as explained above and the family member or community device 5000B is an example of an MCI RN 200.

Numerous modifications and variations of the present disclosure are possible in light of the above teachings. It is therefore to be understood that within the scope of the appended claims, the disclosure may be practiced otherwise than as specifically described herein.

In so far as embodiments of the disclosure have been described as being implemented, at least in part, by software-controlled data processing apparatus, it will be appreciated that a non-transitory machine-readable medium carrying such software, such as an optical disk, a magnetic disk, semiconductor memory or the like, is also considered to represent an embodiment of the present disclosure.

It will be appreciated that the above description for clarity has described embodiments with reference to different functional units, circuitry and/or processors. However, it will be apparent that any suitable distribution of functionality between different functional units, circuitry and/or processors may be used without detracting from the embodiments.

Described embodiments may be implemented in any suitable form including hardware, software, firmware or any combination of these. Described embodiments may optionally be implemented at least partly as computer software running on one or more data processors and/or digital signal processors. The elements and components of any embodiment may be physically, functionally and logically implemented in any suitable way. Indeed the functionality may be implemented in a single unit, in a plurality of units or as part of other functional units. As such, the disclosed embodiments may be implemented in a single unit or may be physically and functionally distributed between different units, circuitry and/or processors.

Although the present disclosure has been described in connection with some embodiments, it is not intended to be limited to the specific form set forth herein. Additionally, although a feature may appear to be described in connection with particular embodiments, one skilled in the art would recognize that various features of the described embodiments may be combined in any manner suitable to implement the technique.

Embodiments of the present technique can generally described by the following numbered clauses:
(1)

A system for determining an indicator value of cognitive impairment in a user comprising circuitry configured to:
receive motion data from a wearable device worn by the user during a time period comprised of a first time period and a second time period, the second time period being after the first time period;
compare the motion data received during the second time period with stored reference motion data, the stored reference motion data representing reference motion carried out when performing a predetermined task during the second period of time;
determine the predetermined task based upon the comparison;
compare the motion data received during the first time period with further stored reference motion data, the further stored reference motion data representing reference motion carried out when performing the determined predetermined task during the first time period; and
determine the indicator value of the cognitive impairment based upon a comparison between the motion data received during the first time period with the further reference motion data.
(2)

A system according to (1) wherein the circuitry is configured to capture the motion data periodically over said time period; and determine the indicator value of the cognitive impairment using the periodically captured motion data.
(3)

A system according to (1) or (2), wherein the motion data is one of time-dependent acceleration or velocity and the circuitry is configured to determine the indicator value of the cognitive impairment by comparing the midpoint errors of the time-dependent acceleration or velocity received during the first time period and the further reference motion data.
(4)

A system according to any one of (1) to (3), wherein the circuitry is further configured to determine the indicator value of the cognitive impairment using physiological data of the individual captured from a second wearable device worn by the user.
(5)

A system according to (4), wherein the physiological data is a myoelectric signature.
(6)

A system according to any one of (1) to (5), wherein the circuitry is configured to receive motion data from a second, different, wearable device and to determine a physical impairment from the motion data received from the second wearable device.
(7)

A system according to any one of (1) to (6), wherein the reference motion data is captured from the individual over a time longer than the time period.
(8)

A system according to any one of (1) to (6), wherein the reference motion data is captured from healthy individuals.
(9)

A system according to (8), wherein the healthy individuals are the same demographic as the user.
(10)

A system according to any one of (1) to (9), wherein the circuitry is configured to correlate the indicator value of the cognitive impairment with other indicator values for the user; and in the event that the correlation is below a threshold, issuing a notification.
(11)

A system according to (10), wherein the notification is issued to one or both of the user and a medical practitioner.
(12)

A system for notifying cognitive impairment in a user, the system comprising circuitry configured to: receive the indicator value of the cognitive impairment from a system according to any one of (1) to (9); and issue a notification based on the indicator value of the cognitive impairment.
(13)

A method for determining an indicator value of cognitive impairment in a user comprising:
receiving motion data from a wearable device worn by the user during a time period comprised of a first time period and a second time period, the second time period being after the first time period;
comparing the motion data received during the second time period with stored reference motion data, the stored reference motion data representing reference motion carried out when performing a predetermined task during the second period of time;
determining the predetermined task based upon the comparison;
comparing the motion data received during the first time period with further stored reference motion data, the further stored reference motion data representing reference motion carried out when performing the determined predetermined task during the first time period; and
determining the indicator value of the cognitive impairment based upon a comparison between the motion data received during the first time period with the further reference motion data.
(14)

A computer program comprising computer readable instructions which, when loaded onto a computer, configures the computer to perform a method according to (13).

### CITATION LIST

### Non Patent Literature

NPL 1: Zhou, H.; Lee, H.; Lee, J.; Schwenk, M.; Najafi, B. Motor Planning Error: Toward Measuring Cognitive Frailty in Older Adults Using Wearables. Sensors 2018, 18, 926. https://doi.org/10.3390/s18030926

1. A system for determining an indicator value of cognitive impairment in a user comprising circuitry configured to:
receive motion data from a wearable device worn by the user during a time period comprised of a first time period and a second time period, the second time period being after the first time period;
compare the motion data received during the second time period with stored reference motion data, the stored reference motion data representing reference motion carried out when performing a predetermined task during the second period of time;
determine the predetermined task based upon the comparison;
compare the motion data received during the first time period with further stored reference motion data, the further stored reference motion data representing reference motion carried out when performing the determined predetermined task during the first time period; and
determine the indicator value of the cognitive impairment based upon a comparison between the motion data received during the first time period with the further reference motion data.

2. A system according to claim 1 wherein the circuitry is configured to capture the motion data periodically over said time period; and
determine the indicator value of the cognitive impairment using the periodically captured motion data.

3. A system according to claim 1, wherein the motion data is one of time-dependent acceleration or velocity and the circuitry is configured to determine the indicator value of the cognitive impairment by comparing the midpoint errors of the time-dependent acceleration or velocity received during the first time period and the further reference motion data.

4. A system according to claim 1, wherein the circuitry is further configured to determine the indicator value of the cognitive impairment using physiological data of the individual captured from a second wearable device worn by the user.

5. A system according to claim 4, wherein the physiological data is a myoelectric signature.

6. A system according to claim 1, wherein the circuitry is configured to receive motion data from a second, different, wearable device and to determine a physical impairment from the motion data received from the second wearable device.

7. A system according to claim 1, wherein the reference motion data is captured from the individual over a time longer than the time period.

8. A system according to claim 1, wherein the reference motion data is captured from healthy individuals.

9. A system according to claim 8, wherein the healthy individuals are the same demographic as the user.

10. A system according to claim 1, wherein the circuitry is configured to correlate the indicator value of the cognitive impairment with other indicator values for the user; and in the event that the correlation is below a threshold, issuing a notification.

11. A system according to claim 10, wherein the notification is issued to one or both of the user and a medical practitioner.

12. A system for notifying cognitive impairment in a user, the system comprising circuitry configured to: receive the indicator value of the cognitive impairment from a system according to claim 1; and issue a notification based on the indicator value of the cognitive impairment.

13. A method for determining an indicator value of cognitive impairment in a user comprising:
receiving motion data from a wearable device worn by the user during a time period comprised of a first time period and a second time period, the second time period being after the first time period;
comparing the motion data received during the second time period with stored reference motion data, the stored reference motion data representing reference motion carried out when performing a predetermined task during the second period of time;
determining the predetermined task based upon the comparison;
comparing the motion data received during the first time period with further stored reference motion data, the further stored reference motion data representing reference motion carried out when performing the determined predetermined task during the first time period; and
determining the indicator value of the cognitive impairment based upon a comparison between the motion data received during the first time period with the further reference motion data.

14. A computer program comprising computer readable instructions which, when loaded onto a computer, configures the computer to perform a method according to claim 13.
